(19) United States
(12) Patent Application Publication
(10) Pub. No.: US 2023/0190131 A1
(43) Pub. Date: Jun. 22, 2023

(54) DERIVATION OF CARDIAC AND RESPIRATORY SIGNALS FROM A THERMAL CAMERA SYSTEM

(71) Applicant: Covidien LP, Mansfield, MA (US)

(72) Inventors: Philip C. SMIT, Hamilton (GB); Paul S. ADDISON, Edinburgh (GB); Dean MONTGOMERY, Edinburgh (GB)

(21) Appl. No.: 17/929,545

(22) Filed: Sep. 2, 2022

Related U.S. Application Data
(60) Provisional application No. 63/292,060, filed on Dec. 21, 2021.

(51) Int. Cl. A61B 5/08; A61B 5/00; A61B 5/0205

(52) U.S. Cl. CPC A61B 5/0816; A61B 5/726; A61B 5/0205; A61B 5/7278

(57) ABSTRACT

A thermal camera system is used to derive cardiac and respiratory signals or information via extraction of cardiac and/or respiratory signals from a thermal signal at a region of interest (ROI), by a peak picking analysis of the thermal signal, or via analysis of a wavelet transform of the thermal signal.

### CROSS-REFERENCE TO RELATED APPLICATIONS

The present application claims benefit of priority to U.S. Provisional Patent Application No. 63/292,060, entitled “DERIVATION OF CARDIAC AND RESPIRATORY SIGNALS FROM A THERMAL CAMERA SYSTEM” and filed on Dec. 21, 2021, which is specifically incorporated by reference herein for all that it discloses or teaches.

### FIELD

The present technology is generally related to derivation of cardiac and respiratory signals using a thermal camera system, for example for patient monitoring.

### BACKGROUND

As the need for more remote patient monitoring has grown, not least due to the COVID-19 pandemic, there is a requirement for more technologies to record patient vital signs automatically, continuously and robustly without touching them, or even without staff going into the same room as the patient.

Further, temperature is a key vital sign in the assessment of patients.

Some methods have been known to generate a breathing signal from a thermal video and its use to produce a respiratory rate have focused on the nasal region of the face where cooling and heating of the skin around the nostril leads to a changing temperature over time synchronous with respiration (e.g., Cho, Y., Julier, S. J., Marquardt, N., & Bianchi-Berthouze, N. (2017). Robust tracking of respiratory rate in high-dynamic range scenes using mobile thermal imaging. Biomedical optics express, 8(10), 4480-4503. However, this has a critical failure mode in that the nose may be obscured in the image, e.g., if the patient is under covers or lying prone with the nose not visible in the scene. This method can work well, but only as long as the nostril is visible within the image and can be tracked over time.

What is needed in the art are techniques for additional patient monitoring, with an emphasis on robust and accurate determination of patient temperature, with further reliable determination of cardiac and respiratory signals therefrom.

### SUMMARY

The techniques of this disclosure generally relate to derivation of cardiac and respiratory signals using a thermal camera system.

In exemplary embodiments, a method for determining respiratory or cardiac patient information from a thermal camera, includes: providing at least one thermal camera with a field of view oriented towards a patient; with the at least one thermal camera, determining thermal camera data relating to the patient, including a measured temperature of a patient in at least one region of interest (ROI) over time; with a processor, receiving such thermal camera data as a thermal signal; extracting thermal information across the at least one ROI; aggregating thermal information within the at least one ROI; filtering out respiratory or cardiac signals from the thermal signal; and determining at least one respiratory or cardiac value from the filtered out respiratory or cardiac signals.

In further exemplary aspects, a method for determining respiratory or cardiac patient information from a thermal camera, includes: providing at least one thermal camera with a field of view oriented towards a patient; with the at least one thermal camera, determining thermal camera data relating to the patient, including: a measured temperature of a patient in at least one region of interest (ROI) over time; with a processor, performing a peak picking algorithm on the thermal signal; determining breathing rate from inter-breath periods; and determining respiration rate of the patient.

In further exemplary aspects, a method for determining respiratory or cardiac patient information from a thermal camera, includes: providing at least one thermal camera with a field of view oriented towards a patient; with the at least one thermal camera, determining thermal camera data relating to the patient, including: a measured temperature of a patient in at least one region of interest (ROI) over time; with a processor: extracting the thermal signal; performing a wavelet transform of the thermal signal; identifying respiratory or cardiac bands in the wavelet transform; and determining heart rate or respiration rate of the patient.

In further exemplary aspects, determined respiratory and/or cardiac information are displayed on a screen/monitor.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a diagram showing an exemplary system for patient monitoring using a thermal camera;

FIG. 2 is a chart illustrating exemplary determination of patient core temperature utilizing a thermal camera;

FIG. 3A is an exemplary thermal scan of a patient;

FIG. 3B is an exemplary region of interest (ROI) for a patient;

FIG. 3C is an exemplary ROI for a patient;

FIG. 3D is an exemplary plot of mean temperature signal from an ROI;

FIG. 3E is exemplary plot of mean temperature signal from an ROI;

FIG. 4A is an exemplary thermal scan of a patient;

FIG. 4B is an exemplary region of interest (ROI) for a patient;

FIG. 4C is an exemplary ROI for a patient;

FIG. 4D is an exemplary plot of mean temperature signal from an ROI;

FIG. 4E is exemplary plot of mean temperature signal from an ROI;

FIG. 5A is an exemplary plot a thermal signal for a patient;

FIG. 5B is the respiratory component from the plot of FIG. 5A;

FIG. 5C is the cardiac component from the plot of FIG. 5A;

FIG. 6 is a flowchart of an exemplary method for determining cardiac and respiratory information from a thermal camera;

FIG. 7 is an exemplary diagram of a patient showing movement vectors;

FIG. 8 is an exemplary thermal scan for a patient showing an ROI;

FIG. 9 is an exemplary plot showing peak picking;

FIG. 10 is a plot of an exemplary thermal signal for a patient;

FIG. 11 is an exemplary wavelet transform of the signal of FIG. 10;

FIG. 12 is a flowchart of an exemplary method for determining heart rate (HR) and respiratory rate (RR) from a thermal camera using a wavelet transform; and

FIG. 13 is an exemplary system diagram for a thermal camera system.

### DETAILED DESCRIPTION

As we have noted above, the present disclosure describes derivation of cardiac and respiratory signals using a thermal camera system. Such systems and methods may find application in hospital settings, in general, or in any setting where monitoring one or more patients, e.g., automatically, continuously and/or robustly without touching them, or e.g., without staff going into the same room as the patient, may be beneficial. One exemplary application includes pandemic management, such as for COVID-19 management, in a hospital setting or otherwise.

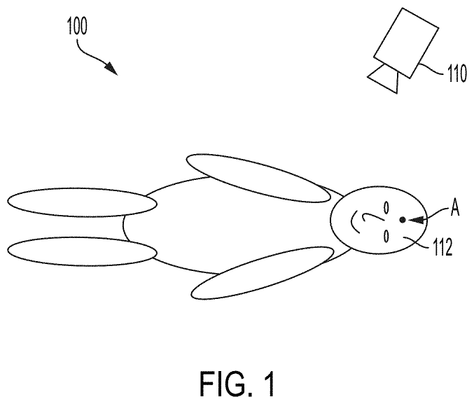

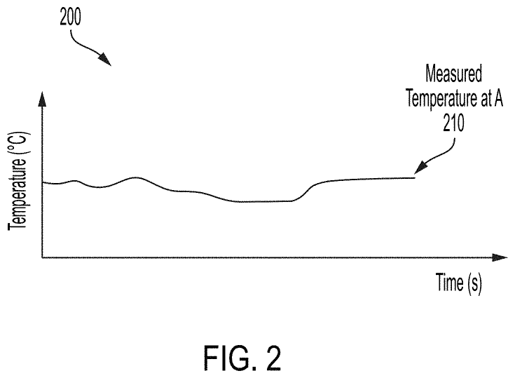

In exemplary embodiments, a thermal camera is configured to take a measurement on a patient location indicative of the core temperature of a patient, e.g., at a point on the surface of a face of the patient. FIG. 1 illustrates, generally at 100, a system and method using a thermal camera 110 providing such a measurement taken at location A on the patient's forehead 112. FIG. 2 illustrates generally at 200, such measured temperature 210 at location A over time, thus providing a continuous measure of the patient's temperature over time.

Exemplary aspects of the present disclosure use thermal video information to provide accurate cardiac and respiratory monitoring of a patient, e.g., heart rate, respiratory rate, etc., as well as providing for measurement, analysis and display of such information over time.

Thermal imaging measures medium and long wave infrared (“MWIR” and “LWIR”) spectral bands. The MWIR band can be of particular importance due to the significant amount blackbody radiation emitted in the band. The camera thus measures the radiation or heat of the body. The present disclosure recognizes that this offers a significant advantage over, for example, RGB and IR imaging since the body produces the emissions/heat and is measured by the thermal camera, whereas the RGB and IR rely on electromagnetic waves in their respective spectral bands to be reflected from the body to be visible. RGB will not work in an unlit room; and IR also requires an IR light source to illuminate the scene.

The present disclosure recognizes that a further advantage that thermal camera systems offer is because heat is emitted from the body, such that the imaging result is invariant to the orientation of the body relative to the camera. In the case of RGB for example, the location of the emissions source (i.e. the light source(s)) and the reflective nature of the surface of the body will determine the observed imaging result.

An additional advantage of thermal imaging is that the thermal intensity response is very much bounded when applied within a medical environment. The human body has an upper temperature limit of about 46° C. (115° F.). The background (e.g., a mattress) is typically about 22° C. (71° F.), and is therefore a lower bound. The present disclosure recognizes that the bounded temperature range makes it much easier from an algorithmic point of view to process and analyze the scene, unlike RGB and IR imaging, which have no such guarantees.

Additionally, the change in temperature, as measured by the thermal camera, is drastic when moving from the skin to the cooler background, thus offering good contrast between the body and background. This makes it easy to detect body edges and as we will show later, movement/modulation of edges contain a lot of physiological information. Good body/background contrast is not necessarily the case with RGB and IR.

Additionally, thermal can, to some extent, “see” through sheets. It is thus easier to detect the body from an algorithmic point of view.

As we have noted above regarding FIGS. 1 and 2, an exemplary method (and system and apparatus) includes monitoring a patient using a thermal camera (e.g., camera 110 in FIG. 1) and take a point on the surface of the face (e.g., location A in FIG. 1) as indicative of the core temperature of the patient. By monitoring the temperature of point A, the exemplary aspects of the present disclosure derive a continuous measure of the patient's temperature over time (as in FIG. 2). We also note that temperature images from thermal cameras may be used for more than patient temperature, core or otherwise, but also e.g., to monitor heat curves of a patient entering and leaving a bed, to provide an indication of their form under sheets, etc.

In exemplary embodiments described herein, thermal video information is assessed to provide accurate heart rate and respiratory rate monitoring of a patient. In addition, the methods provide for a cardiac and respiratory signal over time for display on a monitor screen or for other further processing or use.

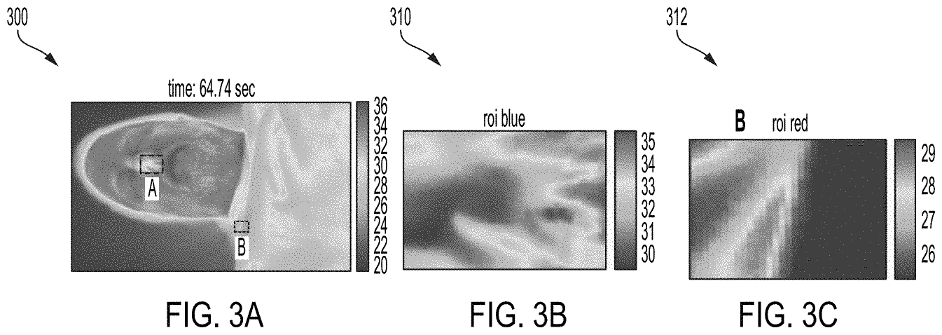

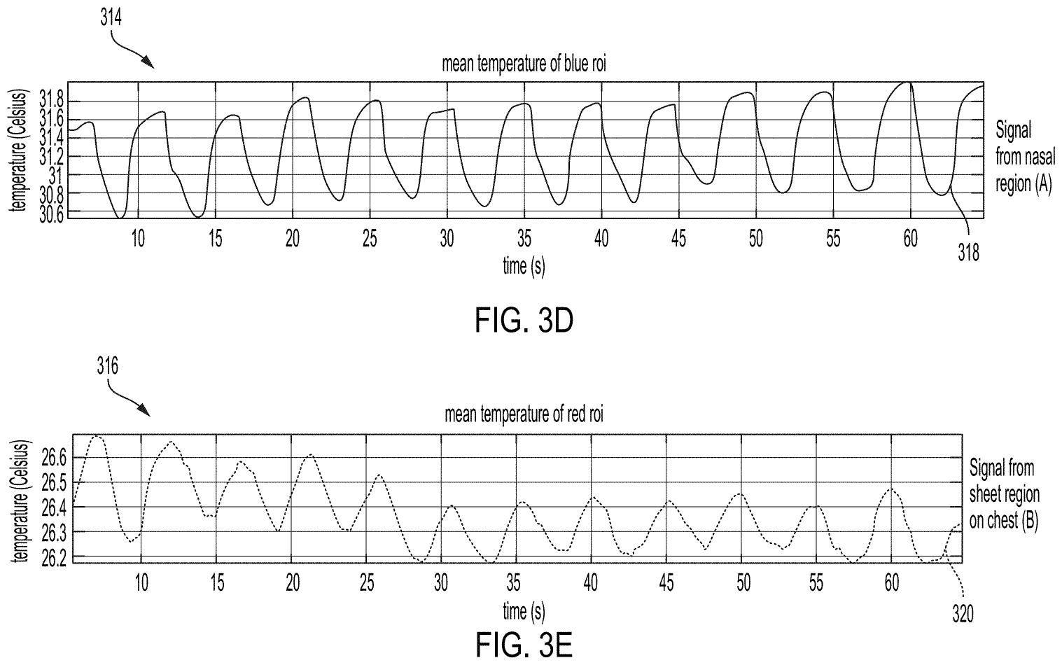

One exemplary respiratory waveform produced by thermal information around the nostril is shown in FIG. 3A (top signal pilot, marker (A), shown generally at 300). The respiration cycle causes the temperature of the nostril region to change with time as heat is exchanged between the nostril region and the environment during the flow of air into and out of the lungs. FIG. 3B shows, generally at 310, an ROI (blue, over approximately 60 seconds) for location (A) within FIG. 3A.

In exemplary embodiments, instead of locating the nostril and analyze changes in temperature due to respiration, various regions of interest (ROIs) may also be located in the thermal image on the patient (e.g., location (B) in FIG. 3A), and alternatively monitoring of this and other possible ROIs for movement over time. FIG. 3C shows, generally at 312, an ROI (red, over approximately 60 seconds) for location (B) within FIG. 3A.

In exemplary embodiments, a focus on or selection of ROIs may relate to those containing an edge or distinct temperature gradient. An example of a respiratory signal generated by focusing on an edge region is shown in FIG. 3A, e.g, region (B), FIG. C, as in this way a respiratory signal can be generated without the need to locate the nostril region.

In general, it may be noted that the ROI containing the edge (edge-ROI) may be located on the skin, clothing, or sheets of the patient. In FIG. 3C (lower signal plot, location (B)), the ROI is actually located on the sheet. This emphasizes the robustness of the method and the ability to generate a signal even if no patient skin can be seen in the image.

It should be noted that the signal generated by the edge-ROI in FIG. 3C (location (B)) contains cardiac information which is also the result of small movements (caused by the cardioballistic action). FIG. 3D and FIG. 3E show signals 318 (temperature (Celsius) vs. mean temperature of blue ROI), 320 (temperature (Celsius) vs. mean temperature of red ROI) from ROI(s) (A) and (B), respectively generally at 314, 316, for those ROIs (A) and (B) shown in FIG. 3A, signal 318 being from the nasal region; and signal 320 being from the sheet region on the chest.

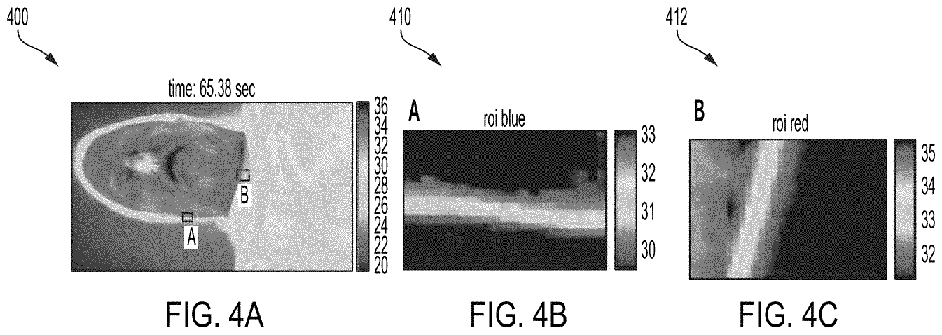

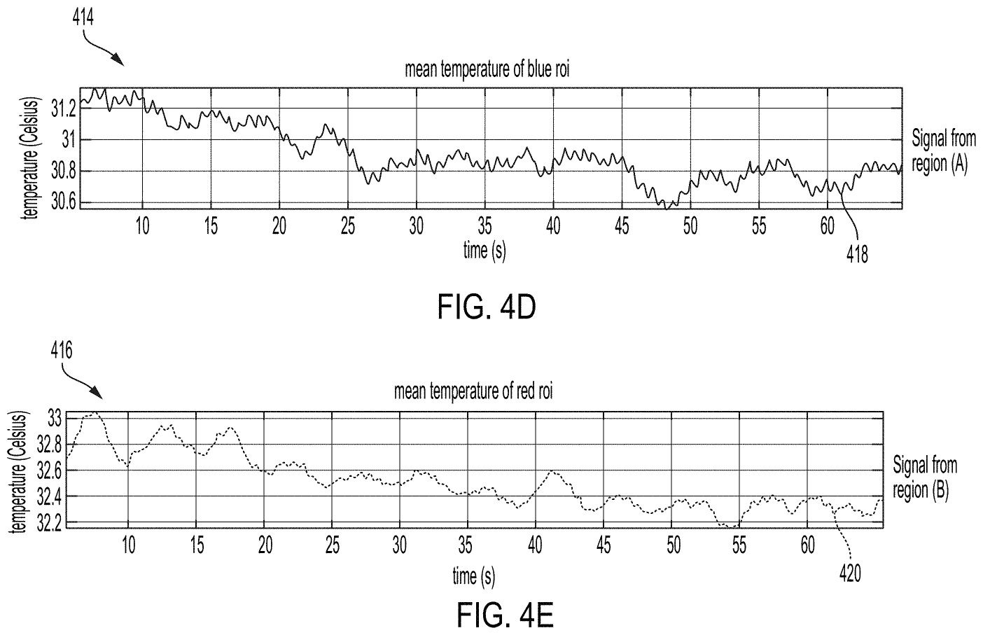

FIG. 4A illustrates, generally at 400, another two examples of edge-ROI generated signals (A) and (B), with cardiac information (cardiac pulses) prominent in these signals. FIG. 4B shows, generally at 410, an ROI (blue, over approximately 60 seconds) for location (A) within FIG. 4A. FIG. 4C shows, generally at 412, an ROI (red, over approximately 60 seconds) for location (B) within FIG. 4A. FIG. 4D and FIG. 4E show signals 418 (temperature (Celsius) vs. mean temperature of blue ROI), 420 (temperature (Celsius) vs. mean temperature of red ROI) from ROI(s) (A) and (B), respectively generally at 414, 416, for those ROIs (A) and (B) shown in FIG. 4A, signal 418 being from a side region of the head; and signal 420 being from the sheet region on the chest.

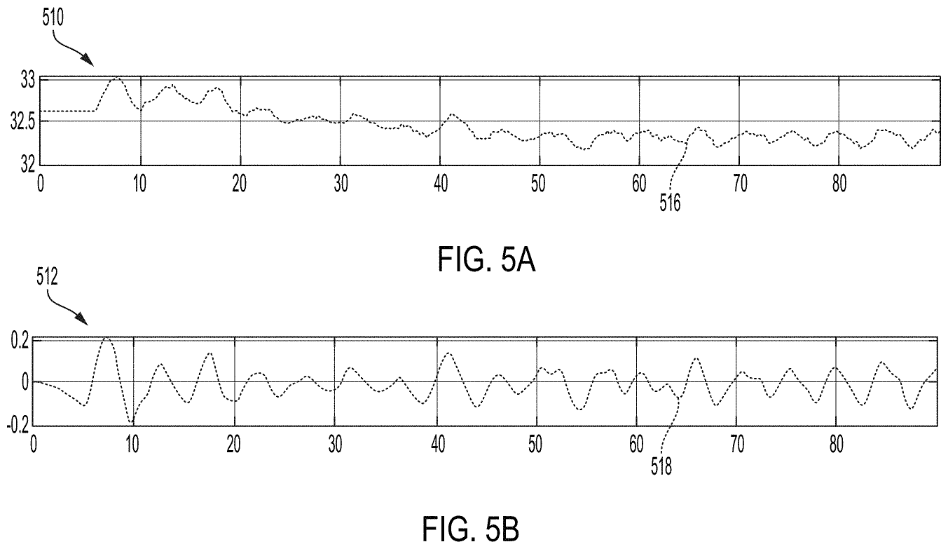

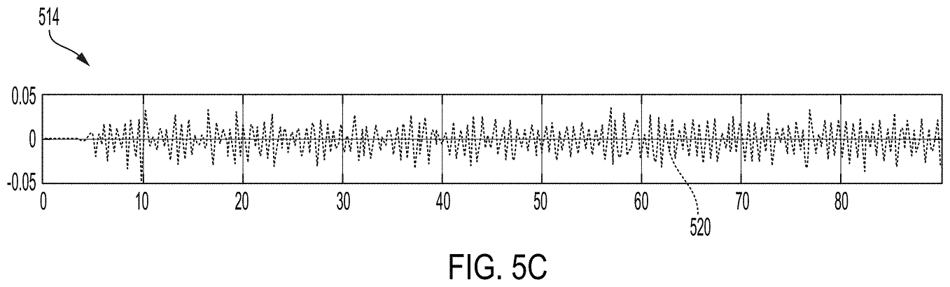

In exemplary embodiments, the cardiac information and respiratory signal information are filtered out separately from these signals to generate separate respiratory and cardiac waveforms for display on a medical monitoring device. This is shown, for example generally at 510, 512 and 514, in FIGS. 5A-C.

A signal 516 generated from the method described above is shown in plot A. the respiratory component 518 (plot B) and cardiac component 520 (plot C) are also shown. These were derived by band-pass filtering the original signal 516 (plot A). In exemplary embodiments, the respiratory and cardiac waveform may be displayed separately on a device, such as a monitor.

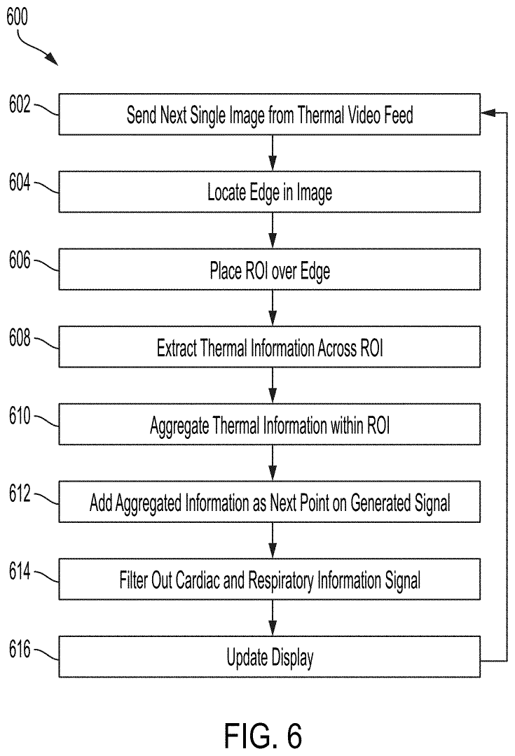

A flow diagram of an exemplary embodiment is shown in FIG. 6 for generation of a respiratory (and/or cardiac) waveform. At block 602, the next (or first) single image is sent from the thermal video feed, e.g., to a processor or user terminal. At block 604, the edge is located (either automatically or at least partially manually). At block 606, the region of interest (ROI) is placed over the edge (either automatically or at least partially manually). At block 608, thermal information is extracted across the ROI. At block 610, thermal information is aggregated within the ROI. At block 612, aggregated information is added as a next point on the generated signal. At block 614, cardiac and respiratory information signals are filtered out. At block 616, the display is updated and the process returns to block 602.

Note that, in the above example, the temperature is aggregated across the ROI to generate the waveform. However, the present disclosure contemplates other methods for generating a waveform. Accordingly, another exemplary embodiment includes aggregating only the information at select points within the ROI, e.g., just the edge pixels.

Additionally, the waveforms from FIGS. 5A-C were generated by calculating the mean (aggregate) temperature of the ROI. Since movement is the instigator of the signals (except in the case of the nose measurement), exemplary embodiment described herein provide for algorithmically processing the ROI by calculating the centroid (or any other related method) of the ROI for each frame. The centroid movement (expressed in pixels) may then be plotted over time.

The centroid has an x and y component. In further exemplary embodiments, the centroid movement is calculated relative to an arbitrary point (i.e. the Euclidian distance) In further exemplary embodiments, the principle component is extracted, with determination of the movement relative to the mean centroid location and the direction of maximum variance. Additional exemplary embodiments also contemplate any other processing to enhance the signal quality.

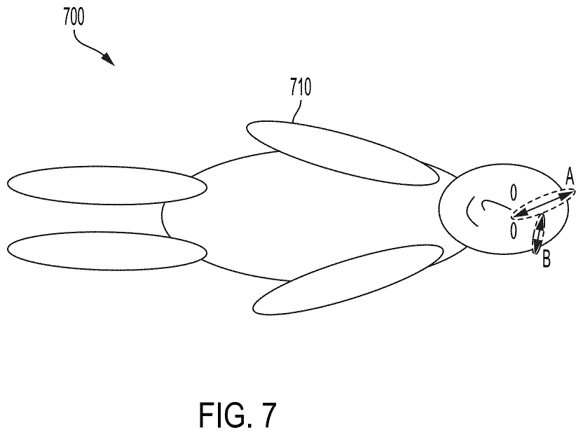

In further exemplary embodiments, different centroid processing can also be applied to extract the respiration and cardiac signals since the forces causing the movements are different in nature. For example, respiration's principal axis of movement may be in direction A as shown in FIG. 7, which generally shows at 700 a prone patient 710, whereas the cardiac movement may be in direction B. The breathing and cardiac signals will therefore be enhanced by considering the direction of each source's movement.

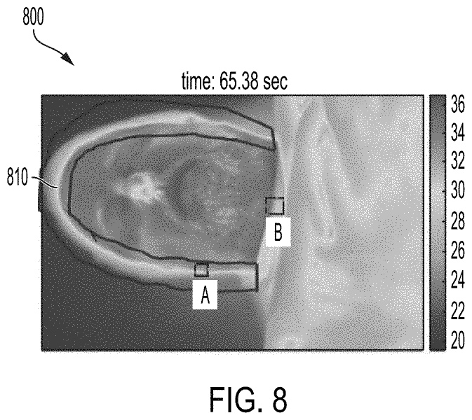

Exemplary methods described herein may also be extended to use all portions of the image that contain a steep temperature gradient. For example, plural areas (or all of the areas) with a large gradient can be located using any appropriate measure (e.g., using a Laplacian filter, or a Sobel filter, etc.). This determination may be made according to a threshold value or values for such gradient, values above which will be considered large. In exemplary aspects, such threshold value or values may be predetermined. For example, FIG. 8 shows generally at 800 thermal information for a patient, as in FIG. 4A, with identified areas of a large gradient within area 810.

In exemplary embodiments, once regions of large gradient are identified (e.g., as in FIG. 8), this area can be dilated and used as an ROI. Any additional methods described herein can then be applied to this irregularly shaped ROI. Alternatively in exemplary embodiments, this ROI may be chopped up into two or more segments, with each segment having an associated waveform for analysis.

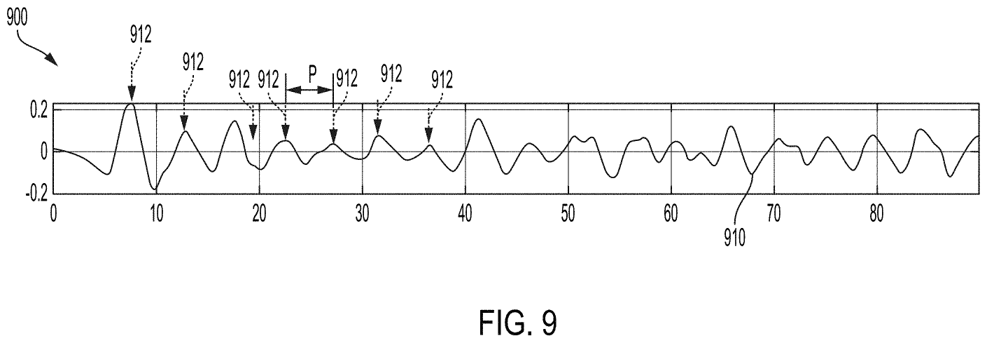

Further exemplary techniques for determining heart rate (HR) and respiration rate (RR) by analysis of the respiratory and cardiac signals are illustrated generally at 900, in FIG. 9 (we note that in this example, signal 910 corresponds to signal 518 in FIG. 5B). With reference to FIG. 9, in exemplary embodiments, an analysis of the respiratory and cardiac signals may be done to determine RR and HR, e.g., by performing a peak picking algorithm on the signal. In exemplary aspects, an example of a number of peaks 912 are located for the respiratory waveform. Once the peaks 912 are located, the inter-breath periods, ‘P’, can be found, from which a breathing rate can be determined. In exemplary embodiments, this may be an instantaneous rate from each breath using the formula:

RR=(1/P)*60, where RR is in breathes per minute (brpm).

An exemplary method averages over a number of breaths to get an average P to use in the above formula, or averages of a number of instantaneous RRs to get an average RR over a period (e.g., over 1 minute).

In further exemplary embodiments, erroneous peaks may be filtered out in the signal before calculating inter-beat periods P.

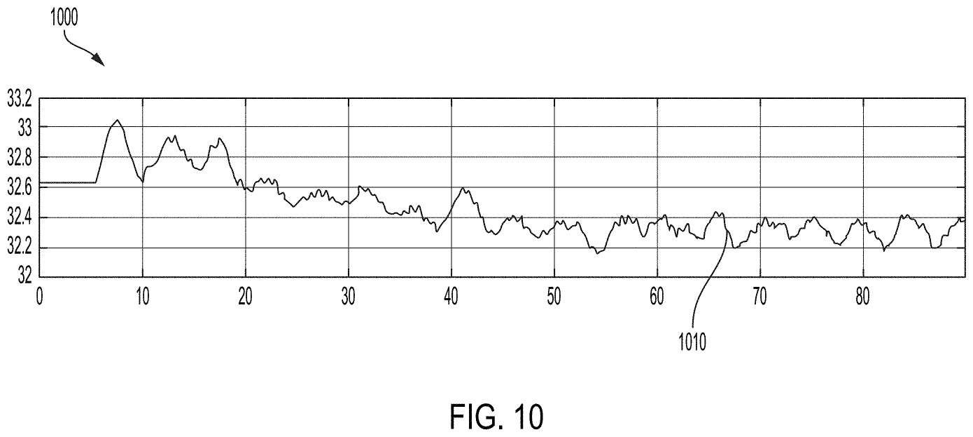

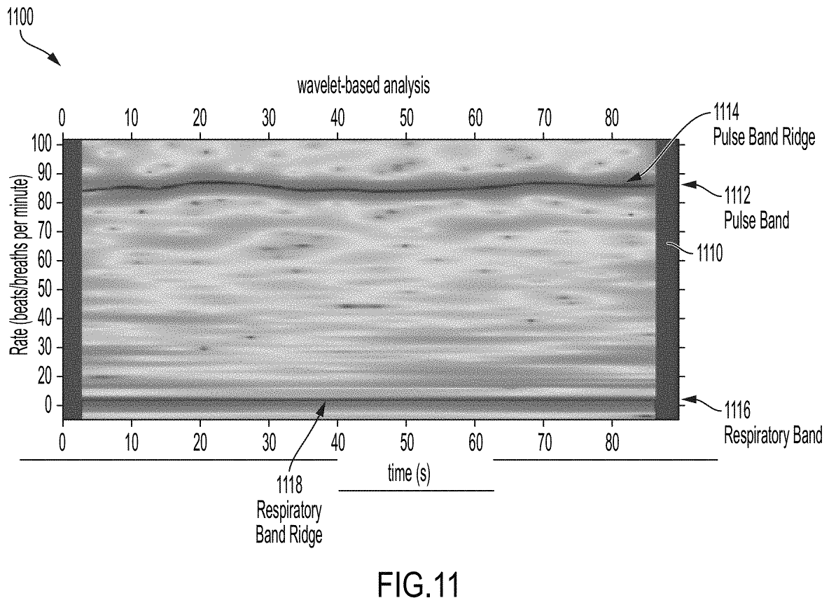

In an alternative exemplary method, a transform of an extracted thermal signal is performed, e.g., the extracted thermal signal shown in FIG. 5A, to produce a representation more amenable to analysis. For example, a wavelet transform 1110 of the thermal signal (in this case signal 1010 of the thermal signal shown generally at 1000 in FIG. 10) may be performed, such as is shown generally at 1100 in FIG. 11. Wavelet transforms are particularly good at separating out pulsatile components from noise.

The wavelet transform 1100, T(a,b) of a signal (t) is given by:

$$T ( a , b ) = \frac{1}{\sqrt{a}} {\int }_{- ∞}^{+ ∞} x ( t ) {ψ}^{*} ( \frac{t - b}{a} ) dt ,$$

where ψ*(t) is the complex conjugate of the wavelet function ψ(t), a is the dilation or scale parameter of the wavelet, b is the location parameter of the wavelet and x(t) is the signal under investigation.

An exemplary method may convert from scale to a characteristic frequency [1] and thus plot a time frequency representation of the wavelet transform such as that shown in FIG. 11. By performing a wavelet transform distinct cardiac and respiratory bands may be observed in wavelet space, for example the pulse band 1112 and the pulse band ridge 1114, and the respiratory band 1116 and the respiratory band ridge 1118 in FIG. 11, where the modulus of the complex wavelet transform of the thermal signal 1010 is plotted.

In exemplary embodiments, the rate (HR or RR) may be extracted from these bands using a method of tracking the ridge in wavelet space. These ridge tracking methods involve following the peak energy of the band over time—i.e., the locus of the maxima on the surface relative to frequency (here RR).

As an example, a ridge 1118 can be seen superimposed as a black line on the respiratory band in FIG. 11. The value of RR can be read directly from the ridge.

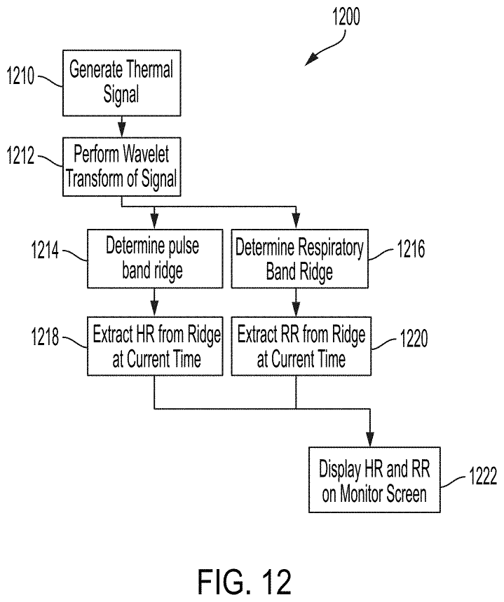

A flow diagram of an example embodiment is shown generally at 1200 in FIG. 12. At block 1210, a thermal signal is generated. At block 1212, a wavelet transform is performed for the signal. At block 1214, the pulse band ridge is determined; and at block 1216, the respiratory band ridge is determined. At block 1218, HR is extracted from the pulse band ridge (e.g., at a current time); and at block 1220, RR is extracted from the respiratory band ridge (e.g., at a current time). At block 1222, HR and RR are displayed on a monitor screen.

In further exemplary embodiments, other transforms may be used in the method including other time-frequency transforms, such as the short-time Fourier transform.

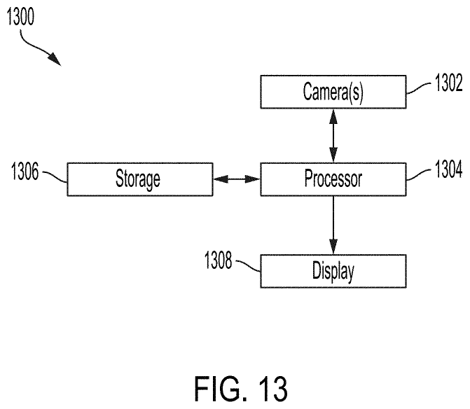

FIG. 13 provides a system diagram shown generally at 1300 and including at least one thermal camera 1302 that is configured to detect thermal data from patients. The data and information from the thermal camera(s) is received and transmitted (via wired or wireless transmission) to a processor 1304 (as part of a computing system, server, etc.), which is configured to process the thermal data, as has been described herein. In exemplary embodiments, the processor is also configured to provide such processing over time. The processor is optionally also configured to provide control functions for the camera(s). Data from the camera(s) and/or from processor calculations is optionally stored in memory/storage 1306. Additionally, data from the camera(s) and/or from processor calculations is optionally transmitted for display on the display device/monitor 1308, as has been described herein.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method for determining respiratory or cardiac patient information from a thermal camera, comprising:
providing at least one thermal camera with a field of view oriented towards a patient;
with the at least one thermal camera, determining thermal camera data relating to the patient, including: a measured temperature of a patient in at least one region of interest (ROI) over time;
with a processor:
receiving such thermal camera data as a thermal signal;
extracting thermal information across the at least one ROI;
aggregating thermal information within the at least one ROI;
filtering out respiratory or cardiac signals from the thermal signal; and
determining at least one respiratory or cardiac value from the filtered out respiratory or cardiac signals.

2. A method in accordance with claim 1, further comprising determining at least one respiratory and at least one cardiac value from filtered out respiratory and cardiac signals.

3. A method in accordance with claim 2, further comprising displaying the filtered out respiratory and cardiac signals on a display.

4. A method in accordance with claim 1, wherein an edge is located in an image from the thermal camera, and further wherein the ROI is placed over the edge.

5. A method in accordance with claim 4, wherein aggregated thermal information within the ROI is added as a next point on the generated signal prior to filtering out cardiac or respiratory information.

6. A method in accordance with claim 1, wherein a temperature gradient is identified in the thermal camera data, with selection of an ROI based upon a threshold value for the thermal gradient.

7. A method in accordance with claim 1, wherein the filtered out respiratory or cardiac signals are determined via bandpass filtering of the original signal.

8. A method in accordance with claim 1, wherein the ROI is algorithmically processed by calculating the centroid of the ROI for each frame, with plotting of the centroid movement over time.

9. A method in accordance with claim 1, wherein centroid processing use used to extract the respiration and cardiac signals from the thermal signal.

10. A method for determining respiratory or cardiac patient information from a thermal camera, comprising:
providing at least one thermal camera with a field of view oriented towards a patient;
with the at least one thermal camera, determining thermal camera data relating to the patient, including: a measured temperature of a patient in at least one region of interest (ROI) over time;
with a processor, performing a peak picking algorithm on the thermal signal;
determining breathing rate from inter-breath periods; and
determining respiration rate of the patient.

11. A method in accordance with claim 10, wherein an average of inter-breath periods is used to determine respiration rate.

12. A method in accordance with claim 10, wherein an average of a number of instantaneous respiration rates are used to get an average respiration rate over a period of time.

13. A method in accordance with claim 10, wherein determined respiration rate information is displayed on a screen.

14. A method for determining respiratory or cardiac patient information from a thermal camera, comprising:
providing at least one thermal camera with a field of view oriented towards a patient;
with the at least one thermal camera, determining thermal camera data relating to the patient, including: a measured temperature of a patient in at least one region of interest (ROI) over time;
with a processor:
extracting the thermal signal;
performing a wavelet transform of the thermal signal;
identifying respiratory or cardiac bands in the wavelet transform; and
determining heart rate or respiration rate of the patient.

15. A method in accordance with claim 14, further comprising determining at least one respiratory and at least one cardiac value from wavelet transform.

16. A method in accordance with claim 14, wherein heart rate is extracted from an identified pulse band ridge in the wavelet transform.

17. A method in accordance with claim 16, wherein the pulse band ridge is tracked in wavelet space by following the peak energy of the band over time.

18. A method in accordance with claim 14, wherein respiration rate is extracted from an identified respiratory band ridge in the wavelet transform.

19. A method in accordance with claim 18, wherein the respiratory band ridge is tracked in wavelet space by following the peak energy of the band over time.

20. A method in accordance with claim 14, wherein determined heart rate and respiration rate information are displayed on a screen.
